(19) United States
(12) Patent Application Publication
(10) Pub. No.: US 2022/0398672 A1
(43) Pub. Date: Dec. 15, 2022

(54) AUTO CORRECTION ROOM AND BED AND OBSERVATION CHARGES ALIGNED TO CLINICAL HISTORY

(71) Applicant: CERNER INNOVATION, INC., Kansas City, KS (US)

(72) Inventors: Michael Dale Chapman, Kansas City, KS (US); Jamie Marie Carbery, Kansas City, KS (US); Jody Lynn Taylor, Oviedo, FL (US); Joshua Brasel, Kansas City, KS (US); Paul Cannon, Kansas City, MO (US)

(21) Appl. No.: 17/348,426

(22) Filed: Jun. 15, 2021

(51) Int. Cl. G06Q 40/00; G16H 40/20; G16H 10/60

(52) U.S. Cl. CPC G06Q 40/12; G16H 40/20; G16H 10/60

(57) ABSTRACT

Methods, systems, and computer storage media are provided for auto-correcting a charge event corresponding to clinical history. An auto-correction clinical system includes intelligence circuitry. The auto-correction clinical system is structured to receive accommodation history corresponding to a patient, receive encounter history corresponding to the patient, determine whether the accommodation history includes a gap, determine a charge event corresponding to a patient status indicator and a care period corresponding to one or more days based on the determination of the gap in the accommodation history, and generate an auto-correct event based on the charge event and the care period.

### BACKGROUND

The revenue cycle is the process by which clinical activity is tracked by healthcare systems to manage revenue derived from patients. The revenue cycle generally begins when a patient presents to a healthcare facility or provider. When the healthcare provider receives all payments for the services rendered, the revenue cycle ends. During the revenue cycle, inaccurate charges lead to errors in claims and billing such that payments are delayed or not provided to healthcare providers. In current systems, claims are submitted for orders that may identify inaccurate clinical units or may have mismatches in dates and/or times or missing information between certain aspects of a patient encounter that result in inaccurate charges. To address the inaccurate charges, current revenue cycle systems require human intervention to manually review and edit data entries across multiple clinical systems to correct the charges resulting in time and resource inefficiencies and increased costs.

### BRIEF SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present invention relate to leveraging clinical system data to correct charge(s) incurred during the revenue cycle via an auto-correction system. In particular, the present invention utilizes clinical data to automatically correct a charge event (e.g., a room and bed charge or an observation charge) aligned to clinical history. The accommodation history corresponding to a patient is received at an auto-correction system. The auto-correction system receives the encounter history corresponding to the patient. The auto-correction system then determines whether the accommodation history comprises a gap. If the auto-correction system determines the accommodation history comprises a gap, a charge event corresponding to a patient status indicator and a care period corresponding to one or more days is determined. An auto-correct event is generated via the auto-correction system based on the charge event and the care period.

### BRIEF DESCRIPTION OF THE SEVERAL VIEWS OF THE DRAWINGS

The present invention is described in detail below with reference to the attached drawing figures, wherein:

FIG. 1 is a block diagram of an exemplary computing environment suitable for use in implementing the present disclosure;

FIG. 2 is a block diagram of an exemplary system for auto-correcting a charge event corresponding to clinical history, in accordance with an embodiment of the present disclosure;

FIG. 3 is a block diagram of an exemplary implementation of an auto-correction clinical system, in accordance with some embodiments of the present disclosure;

FIG. 4 is a flow diagram showing an exemplary method for auto-correcting a charge event corresponding to clinical history, in accordance with various embodiments of the present disclosure; and

FIG. 5 is a flow diagram showing an exemplary method for auto correction of a charge event, in accordance with various embodiments of the present disclosure.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

As noted in the Background, current systems allow claims to be submitted for orders that may identify inaccurate clinical units or may have mismatches in dates and/or times or missing information between certain aspects of a patient encounter that result in inaccurate charges. To address the inaccurate charges, current revenue cycle systems require human intervention to manually review and edit data entries across multiple clinical systems to correct the charges resulting in time and resource inefficiencies and increased costs.

Embodiments of the present invention relate to leveraging clinical system data to correct charge(s) incurred during the revenue cycle via an auto-correction system. The present invention utilizes clinical data to automatically correct a charge event (e.g., a room and bed charge or an observation charge) aligned to clinical history. The accommodation history (e.g., the accommodation location history) corresponding to a patient is received at an auto-correction clinical system. The auto-correction clinical system receives encounter history corresponding to the patient. The auto-correction clinical system then determines whether the accommodation history comprises a gap. If the auto-correction clinical system determines the accommodation history comprises a gap, a charge event corresponding to a patient status indicator and a care period corresponding to one or more days is determined. An auto-correct event is generated via the auto-correction clinical system based on the charge event and the care period.

Accordingly, in one aspect, an embodiment is directed to one or more computer storage media having computer-executable instructions embodied thereon that, when executed by a computer, causes the computer to perform operations. The operations include receiving, via an auto-correction clinical system comprising intelligence circuitry, accommodation history corresponding to a patient. The operations also includes receiving, via the auto-correction clinical system, encounter history corresponding to the patient. The operations further include determining, via the intelligence circuitry, whether the accommodation history comprises a gap. The operations also include determining, via the auto-correction clinical system, a charge event corresponding to a patient status indicator and a care period corresponding to one or more days responsive to the determination of the gap in the accommodation history and the encounter history. The operations also include generating, via the auto-correction clinical system, an auto-correct event based on the charge event and the care period.

In another aspect of the invention, an embodiment of the present invention is directed to a method. The method includes include receiving, via an auto-correction clinical system comprising intelligence circuitry, accommodation history corresponding to a patient. The method also includes receiving, via the auto-correction clinical system, encounter history corresponding to the patient. The method further include determining, via the intelligence circuitry, whether the accommodation history comprises a gap. The method also include determining, via the auto-correction clinical system, a charge event corresponding to a patient status indicator and a care period corresponding to one or more days responsive to the determination of the gap in the accommodation history and the encounter history. The method also include generating, via the auto-correction clinical system, an auto-correct event based on the charge event and the care period.

In a further aspect, an embodiment is directed to a computerized system that includes one or more processors and a non-transitory computer storage medium storing computer-useable instructions that, when used by the one or more processors, cause the one or more processors to: receive, via an auto-correction clinical system comprising intelligence circuitry, accommodation history corresponding to a patient, receive, via the auto-correction clinical system, encounter history corresponding to the patient, determine, via the intelligence circuitry, whether the accommodation history comprises a gap, determine, via the auto-correction clinical system, a charge event corresponding to a patient status indicator and a care period corresponding to one or more days responsive to the determination of the gap in the accommodation history and the encounter history, and generate, via the auto-correction clinical system, an auto-correct event based on the charge event and the care period.

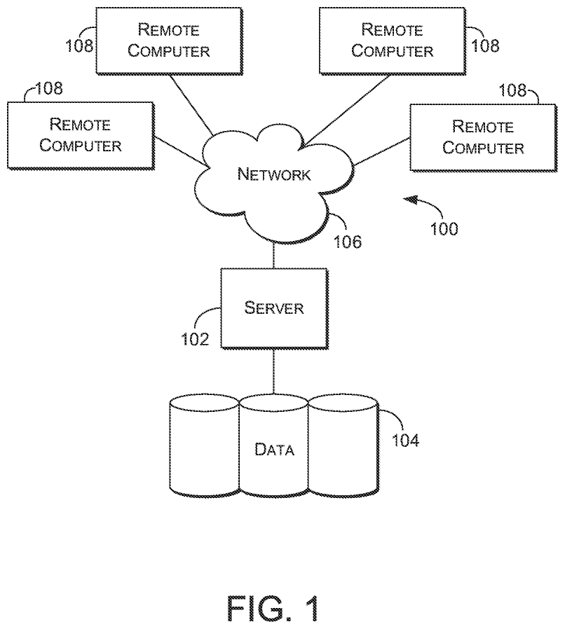

An exemplary computing environment suitable for use in implementing embodiments of the present invention is described below. FIG. 1 is an exemplary computing environment (e.g., medical-information computing-system environment) with which embodiments of the present invention may be implemented. The computing environment is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein.

The present invention might be operational with numerous other purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention might be described in the general context of computer-executable instructions, such as program circuitry, being executed by a computer. Exemplary circuitry (e.g., program circuitry or any other suitable circuitry) comprise circuits (e.g., circuits that include resistors, transistors, capacitors, inductors, diodes, and/or any suitable component whether hardware and/or programmatic code), routines, programs, objects, components, and/or data structures that perform particular tasks or implement particular abstract data types. The present invention might be practiced in distributed computing environments where tasks are performed by remote processing devices that are linked through a communications network. In a distributed computing environment, program circuitry might be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including data store 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of computer-readable media. Computer-readable media can be any available media that might be accessed by control server 102, and includes volatile and nonvolatile media, as well as, removable and nonremovable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program circuitry or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Communication media typically embodies computer-readable instructions, data structures, program circuitry or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 102 might operate in a computer network 106 using logical connections to one or more remote computers 108. Remote computers 108 might be located at a variety of locations in a medical or research environment, including clinical laboratories (e.g., molecular diagnostic laboratories), hospitals and other inpatient settings, ambulatory settings, medical billing and financial offices, hospital administration settings, home healthcare environments, clinicians' offices, Center for Disease Control, Centers for Medicare & Medicaid Services, World Health Organization, any governing body either foreign or domestic, Health Information Exchange, and any healthcare/government regulatory bodies not otherwise mentioned. Clinicians may comprise a treating physician or physicians, specialists such as intensivists, surgeons, radiologists, cardiologists, and oncologists, emergency medical technicians, physicians' assistants, nurse practitioners, nurses, nurses' aides, pharmacists, dieticians, microbiologists, laboratory experts, laboratory technologists, genetic counselors, researchers, students, and the like. The remote computers 108 might also be physically located in nontraditional medical care environments so that the entire healthcare community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program circuitry or portions thereof might be stored in association with the control server 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a pointing device (commonly referred to as a mouse), a trackball, a touch display, or a touch pad. Other input devices include microphones, satellite dishes, scanners, or the like. Commands and information might also be sent directly from a remote healthcare device to the control server 102. In addition to a monitor, the control server 102 and/or remote computers 108 might comprise other peripheral output devices, such as speakers and a printer.

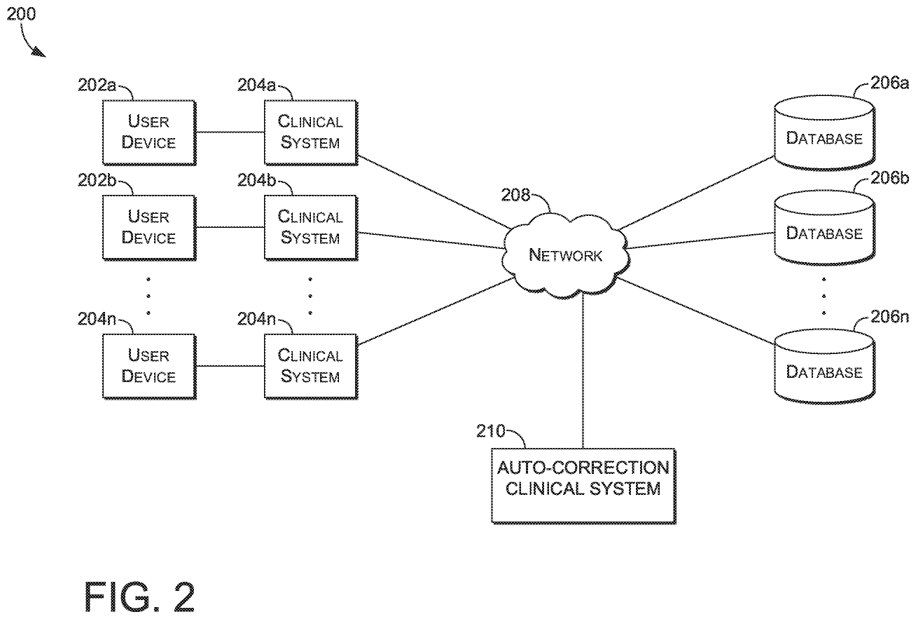

Turning now to FIG. 2, an exemplary clinically driven auto-correction system 200 is depicted suitable for use in implementing embodiments of the present invention. The clinically driven auto-correction system 200 is merely an example of one suitable computing system environment and is not intended to suggest any limitation as to the scope of use or functionality of embodiments of the present invention. Neither should the auto-correction system 200 be interpreted as having any dependency or requirement related to any single system, device, and/or component or combination of systems, devices, and/or components illustrated therein.

The clinically driven auto-correction system 200 includes the user device(s) 202a-202n, clinical system(s) 204a-204n, database(s) 206a-206n, and auto-correction clinical system 210, all in communication with one another via a network 208. The network 208 may include, without limitation, one or more secure local area networks (LANs) or wide area networks (WANs). The network 208 may be a secure network associated with a facility such as a healthcare facility. The secure network may require that a user log in and be authenticated in order to send and/or receive information over the network.

The systems, devices, and/or components illustrated in FIG. 2 are exemplary in nature and in number and should not be construed as limiting. Any number of systems, devices, and/or components may be employed to achieve the desired functionality within the scope of embodiments hereof. Further, the systems, devices, and/or components may be located on any number of servers. By way of example only, the auto-correction clinical system 210 might reside on a server, cluster of servers, or a computing device remote from one or more of the remaining components. Although illustrated as separate systems, the functionality provided by each of these components might be provided as a single system, device, and/or component. The single unit depictions are meant for clarity, not to limit the scope of embodiments in any form.

Components of the clinically driven auto-correction system 200 may include a processing unit, internal system memory, and a suitable system bus for coupling various system components, including one or more data stores for storing information (e.g., files and metadata associated therewith). Components of the clinically driven auto-correction system 200 typically includes, or has access to, a variety of computer-readable media.

It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other systems, devices, and/or components, and in any suitable combination and location. Various functions described herein as being performed by one or more entities may be carried out by hardware, firmware, and/or software. For instance, various functions may be carried out by a processor executing instructions stored in memory.

Each of clinical system 204a-204n includes or has access to infrastructure that is capable of receiving and communicating information for use by, for example, the auto-correction clinical system 210. The information received and communicated in association with each of clinical system 204a-204n may comprise general information used by the auto-correction clinical system 210. Each of the clinical systems 204a-204n may receive data from the user device(s) 202a-202n or other systems (e.g., disparate healthcare systems), which may include any number or type of medical devices that may be utilized to provide or measure patient care to a patient.

Each of the clinical systems 204a-204n includes or has access to infrastructure that is capable of storing electronic health records (EHRs), such as database(s) 206a-206n of patients associated with clinical system(s) 204a-204n. EHRs may comprise electronic clinical documents such as images, clinical notes, orders, summaries, reports, analyses, or other types of electronic medical documentation relevant to a particular patient's condition and/or treatment. Electronic clinical documents contain various types of information relevant to the condition and/or treatment of a particular patient and can include information relating to, for example, patient identification information, images, alert history, culture results, physical examinations, vital signs, past medical histories, surgical histories, family histories, histories of present illnesses, current and past medications, allergies, symptoms, past orders, completed orders, pending orders, tasks, lab results, other test results, patient encounters and/or visits, immunizations, physician comments, nurse comments, other caretaker comments, and a host of other relevant clinical information. In some embodiments, the clinical systems 204a-204n may receive data from health information exchanges (“HIEs”), personal health records (“PHRs”), patient claims, and other health records associated with a patient.

The user device(s) 202a-202n may be any type of computing device used within a healthcare facility or as part of the claims processing process to receive, display, and send information to another user or system. The user device(s) 202a-202n may be capable of communicating via the network with the clinical system(s) 204a-204n, database(s) 206a-206n, or auto-correction clinical system 210. Such devices may include any type of mobile and portable devices including cellular telephones, personal digital assistants, tablet PCs, smart phones, and the like.

The user device(s) 202a-202n is configured to display information to a clinician or user via a display. The information may include communications initiated by and/or received by auto-correction clinical system 210. Embodiments are not intended to be limited to visual display but rather may also include audio presentation, visual presentation, combined audio/visual presentation, and the like.

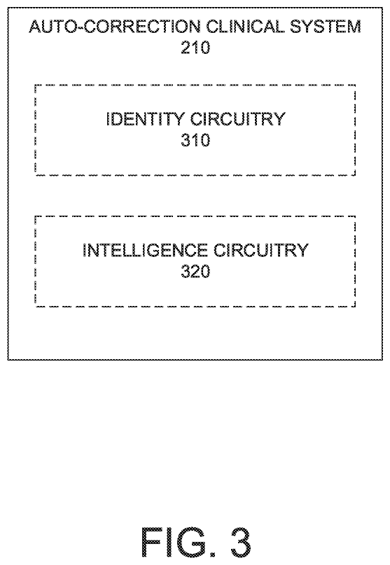

Generally, the auto-correction clinical system 210 is structured to track, monitor, and/or auto-correct charge event(s) corresponding to clinical history. The auto-correction clinical system 210 may track and collect data (e.g., charge data) corresponding to an encounter with a patient at pre-registration, registration, charge capture, coding, claims submission, remittance processing, third-party follow up, patient collections, utilization review, and the like. Although the auto-correction clinical system 210 is depicted as a single system, it is contemplated that each clinical system 204a-204n may utilize a different auto-correction clinical system 210. Further description of the auto-correction clinical system 210 is provided herein with reference to FIG. 3.

FIG. 3 is a block diagram of an exemplary implementation of an auto-correction clinical system according to an example embodiment. The auto-correction clinical system 210 may include one or more circuits, systems, routines, objects, and/or components. In some embodiments, the auto-correction clinical system 210 may include the identity circuitry 310 and/or the intelligence circuitry 320.

Initially, the auto-correction clinical system 210 may be structured to receive the accommodation history corresponding to a patient. The accommodation history may be received in response to clinical activity such as, but not limited to, the pre-registration/registration of a patient, presentation of a patient at a clinical/care facility, direct admit of a patient, an order for a patient during an encounter, and/or any other activity that causes a patient to be identified by the auto-correction clinical system 210. The auto-correction clinical system 210 may identify, via the identity circuitry 310, the clinical activity corresponding to the patient as the patient moves from one clinical unit to another clinical unit. The clinical activity may correspond to one or more encounters associated with the patient. Accordingly, the accommodation history may be received in response to an encounter associated with the patient.

In some examples, the auto-correction clinical system 210 may determine whether the encounter(s) qualifies for inpatient room and bed and/or observation charges. If the encounter(s) qualifies for inpatient room and bed and/or observation charges, the auto-correction clinical system 210 may be structured to receive the accommodation history corresponding to the patient associated with the encounter(s). The auto-correction clinical system 210 may receive or otherwise retrieve the accommodation history corresponding to the patient from the database(s) 206a-206n, memory, clinical system(s) 204a-204n, and/or any other suitable system, database, or server whether directly or indirectly communicatively coupled to the auto-correction system 200.

In some embodiments, the encounter may be associated with encounter history (e.g., one or more previous encounters) corresponding to the patient. In this regard, the auto-correction clinical system 210 may receive, the encounter history corresponding to the patient. The auto-correction clinical system 210 may receive or otherwise retrieve the encounter history corresponding to the patient from the database(s) 206a-206n, memory, clinical system(s) 204a-204n, and/or any other suitable system, database, or server whether directly or indirectly communicatively coupled to the auto-correction system 200.

In some embodiments, the auto-correction clinical system 210 may determine a charge event. For example, the auto-correction clinical system 210 may calculate the charge event(s) that should be applied for each encounter. The charge event may include a room and bed and/or observation charge, observation charge, missing charge, and/or any other suitable charge associated with the clinical activity. The charge event may correspond to a patient status indicator (e.g., a patient status order and/or an adjustment order). As used herein, the “patient status indicator” may refer to a patient status order that includes or otherwise identifies a patient level of care. The patient status indicator is associated with the clinical activity corresponding to a clinical unit. Alternatively or additionally, in some embodiments, the auto-correction clinical system 210 may determine the charge event corresponding to a care period (e.g., a period during which one or more charges for clinical care may be incurred).

In some examples, the auto-correction clinical system 210 may determine a charge event corresponding to the patient status indicator and the care period. The patient status indicator and the care period may correspond to one or more days based on the accommodation history and/or the encounter history. For example, the auto-correction clinical system 210 may calculate the charge event(s) (e.g., one or more inpatient room and bed charge(s)) corresponding to the patient status order and the care period. The auto-correction clinical system 210 may utilize the last inpatient accommodation for each day in the care period (e.g., the charge period) to calculate the charge event(s). Advantageously, the auto-correction clinical system 210 calculates which charge events should be applied for each encounter for each date of service.

In some embodiments, the auto-correction clinical system 210 may split a charge event(s) (e.g. hourly observation charge(s)) across multiple units within a care period (e.g., within the same day). In further examples, the charge event (e.g. observation charge(s)) may be allocated (e.g., automatically allocated) or otherwise associated with one or more clinical units as the auto-correction clinical system 210 monitors the clinical activity associated with the patient. For example, the charge event may be automatically allocated to one or more clinical units as the identity circuitry 310 identifies the clinical activity of the patient as the patient moves from one clinical unit to another clinical unit. The clinical activity may be included (e.g., recorded) in the clinical history associated with the encounter(s). In some examples, the charge event may be adjusted (e.g., rounded) and associated with or otherwise applied to the last unit of a care period (e.g., the last unit of each day).

To determine the charge event, for example, the auto-correction clinical system 210 may utilize the accommodation history (e.g., the previous accommodation history) if present in the encounter history regardless of one or more previous charges. The accommodation history and/or the encounter history may include an encounter such as an inpatient encounter. In some examples, the charge event determined may include a charge event for the last accommodation of the day for the care period (e.g., the entire date of service or any other suitable charge period). Advantageously, the auto-correction clinical system 210 ensures there is not any backdating over the accommodation history (e.g., previous accommodation codes) and/or room and bed and/or observation charges because the care was already delivered.

In some embodiments, the auto-correction clinical system 210 may include the intelligence circuitry 320. The intelligence circuitry 320 may be structured to determine whether the accommodation history includes a gap. The intelligence circuitry 320 may monitor the clinical activity to determine whether the accommodation history includes a gap. A gap may occur when the encounter type for which the intelligence circuitry 320 is charging does not exist in the history for a specific service date.

In some embodiments, the auto-correction clinical system 210, may be structured to determine a charge event corresponding to a patient status indicator and a care period corresponding to one or more days based on the determination of the gap in the accommodation history. For example, if the auto-correction clinical system 210 determines a charge event is needed for a care period (e.g., a date of service) for which there is no corresponding clinical activity, the intelligence circuitry 320 may determine a charge event (e.g., a missing charge) from the accommodation history corresponding to the patient status indicator (e.g., the patient status order that identifies the patient level of care). In some embodiments, the intelligence circuitry 320 may determine the care period (e.g., the charge period), the patient level of care, or a combination thereof from the patient status indicator. The intelligence circuitry 320 may determine the charge event from the accommodation included in the patient status order.

In response to the determination the charge event from the accommodation included in the patient status order, the intelligence circuitry 320 may fill-in (e.g., populates and/or pre-populates) the gap. For example, the intelligence circuitry 320 may fill-in (e.g., pre-populate) the charge event (e.g., the missing charge(s)), the care period (e.g., the charge period), the patient level of care, or a combination thereof. In some embodiments, the charge event may change in response to one or more patient status indicators (e.g., one or more subsequent patient status orders, adjustment orders, etc.). Advantageously, the intelligence circuitry 320 leaves the clinical history intact while determining or otherwise automatically adjusting one or more charges in the auto-correction clinical system 210. In some examples, the intelligence circuitry 320 leaves the clinical history intact while determining or otherwise automatically adjusting one or more charges in the auto-correction clinical system 210 in real-time or near real-time.

In some embodiments, the auto-correction clinical system 210 may generate an auto-correct event. As used herein, the term “auto-correct event” may be used to refer to one or more corrections generated automatically based on one or more charge events determined. The auto-correction clinical system 210 may monitor the clinical activity and the determination of the one or more charge events to generate the auto-correct event. In some examples, the auto-correct event may be based on the one or more charge events determined, a care period, or a combination thereof.

In some embodiments, the generation of the auto-correct event may include a comparison of the charge event determined, or otherwise calculated, to a secondary charge event. The auto-correction clinical system 210 may receive or otherwise retrieve the secondary charge event from the database(s) 206a-206n, memory, clinical system(s) 204a-204n, and/or any other suitable system, database, or server whether directly or indirectly communicatively coupled to the auto-correction system 200. Accordingly, the auto-correction clinical system 210 may compare the charge event(s) (e.g., the inpatient room and bed charge(s)) calculated to one or more secondary charge events (e.g., one or more existing charges).

Alternatively or additionally, the auto-correct event may be determined based on the compared charge event and the secondary charge event according to the care period. The auto-correction clinical system 210 may compare the charge event calculated to the secondary charge event to ensure each interval (e.g., each day, hour, minute, or any other suitable time) within a care period is associated with a charge event. For example, the care period (e.g., length of stay) may be N days (e.g., 10 days) such that the auto-correction clinical system 210 determines whether there is at least one charge event per interval (e.g., per day) resulting in at least 10 charges. If on day 8 of 10 the auto-correction clinical system 210 identifies encounter history corresponding to an encounter associated with a charge event for day 8 and the auto-correction clinical system 210 identifies assisted history (e.g., history information that includes a manual change) associated with a charge event for day 8, the auto-correction clinical system 210 may compare the encounter history and the assisted history to determine whether to utilize or otherwise apply the charge event included within the encounter history or the assisted history. In some examples, if the assisted history is associated with an honored source, the auto-correction clinical system 210 may utilize or otherwise apply the charge event included within the assisted history. If the assisted history is associated with a dishonored source, the auto-correction clinical system 210 may utilize or otherwise apply the charge event included within the encounter history.

In response to the comparison of the charge event determined (e.g., one or more charge events calculated) to the secondary charge event (e.g., one or more existing charge events), the auto-correction clinical system 210 may determine whether there are discrepancies between the charge event determined and the secondary charge event. If there are discrepancies between the charge event determined and the secondary charge event, the auto-correction clinical system 210 may reverse the charge event determined, the secondary charge event, or a combination thereof. Accordingly, the auto-correction clinical system 210 may submit a reversal request (e.g., a request to reverse an encounter, charge event, and/or care period) to reverse the charge event determined.

In some embodiments, the generation of the auto-correct event may include determining, by the auto-correction clinical system 210, the auto-correct event (e.g., a re-charge event) if there are discrepancies between the charge event determined and the secondary charge event. In this regard, the generation of the auto-correct event may include automatically recalculating, reprocessing, and/or re-evaluating one or more charge events (e.g., one or more room and bed and/or observation charges) to correct the encounter.

The auto-correct event may correspond to an encounter associated with the patient status indicator based on the clinical activity for a care period ranging from an interval floor to an interval ceiling. For example, the auto-correct event may correspond to one or more encounters associated with one or more patient status indicators based on the clinical activity (e.g., clinical stays) for a care period (e.g., the most recent 120 days, n number of days prior to the date of discharge, or any other suitable period) ranging from an interval floor (e.g., 1 day) to an interval ceiling (e.g., 120 days).

Alternatively or additionally, the auto-correction clinical system 210 may transmit a post request. The post request may provide or otherwise submit the auto-correct event to the database(s) 206a-206n, memory, clinical system(s) 204a-204n, and/or any other suitable system, database, or server whether directly or indirectly communicatively coupled to the auto-correction system 200. Alternatively or additionally, the auto-correct event may be added to the beginning of the encounter if the care period is over x days (e.g., 120 days) to post an auto-correct event. The post request ensures auto-correct event has been processed up to a specified time frame.

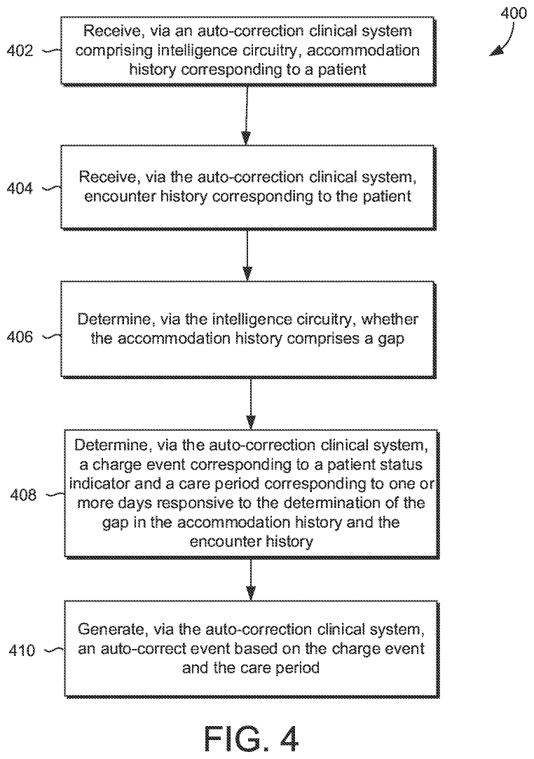

Referring now to FIG. 4 which depicts a flow diagram of an exemplary method 400 for auto-correcting a charge event corresponding to clinical history, in accordance with an embodiment of the present invention. The method may be performed by any computing device (such as computing device described with respect to FIG. 1) with access to an auto-correction system (such as the one described with respect to FIG. 2) or by one or more components of the auto-correction system (such as the auto-correction clinical system, identity circuitry, and/or the intelligence circuitry described with respect to FIGS. 2 and 3).

As shown at 402, the accommodation history corresponding to a patient is received by the auto-correction clinical system. The accommodation history may be received in response to clinical activity (e.g., the direct admit of a patient, receipt of an order for a patient during an encounter, and/or any other activity that causes a patient to be identified by the auto-correction clinical system). In some embodiments, the accommodation history may be retrieved by the auto-correction clinical system in response to the clinical activity. The clinical activity may correspond to one or more encounters associated with the patient. If the encounter(s) qualifies for inpatient room and bed and/or observation charges, the auto-correction clinical system may receive the accommodation history corresponding to the patient associated with the encounter(s).

In some embodiments, the encounter may be associated with encounter history corresponding to the patient. The encounter history may include one or more previous encounters that correspond to a patient. A patient may have one or more encounters that qualifies for receipt and/or use by the auto-correction clinical system. The history of each encounter may be utilized or otherwise determined by the auto-correction clinical system individually for each episode of patient care. For example, patient Jody comes in on Encounter A from care period May 10, 2021, to May 13, 2021. Patient Jody is re-admitted on Encounter B from care period May 15, 2021, to May 30, 2021. Each of patient Jody's Encounters A and B may be received and/or utilized by the auto-correction clinical system. In such examples, the history of Encounter A may be processed separately from the history of Encounter B. In such embodiments, the encounter history corresponding to the patient is received by the auto-correction clinical system at 404. Alternatively or additionally, the encounter history may be retrieved by the auto-correction clinical system.

In some embodiments, the auto-correction clinical system determines whether the accommodation history includes a gap at 406. The intelligence circuitry may monitor the clinical activity to determine whether the accommodation history includes a gap. If, for example, a first encounter type (e.g., a previous encounter type) from the accommodation history does not match a secondary encounter type (e.g., a present encounter type), the intelligence circuitry may determine that the accommodation history includes a gap.

At 408, a charge event corresponding to a patient status indicator and a care period corresponding to one or more days is determined based on the determination of the gap in the accommodation history. If the auto-correction clinical system determines a charge event is needed for a care period for which there is not any corresponding clinical activity, the intelligence circuitry may determine the charge event (e.g., a missing charge) from the accommodation history corresponding to the patient status indicator (e.g., the patient status order). In some embodiments, the intelligence circuitry may determine the charge event from the accommodation included in the patient status order such that the intelligence circuitry fills-in (e.g., populates) the charge event (e.g., the missing charge(s)).

In some embodiments, an auto-correct event is generated by the auto-correction clinical system at 410. The auto-correct event may include one or more corrections generated automatically to one or more charge events. In some examples, the auto-correct event may be based on the one or more charge events determined, a care period, or a combination thereof. In some embodiments, the generation of the auto-correct event may include a comparison of the charge event determined (e.g., calculated) to a secondary charge event (e.g., one or more existing charge events).

In response to the comparison of the charge event determined to the secondary charge event, the auto-correction clinical system may determine whether there are discrepancies between the charge event determined and the secondary charge event. In some embodiments, the generation of the auto-correct event may include automatically recalculating, reprocessing, and/or re-evaluating one or more charge events to correct the encounter.

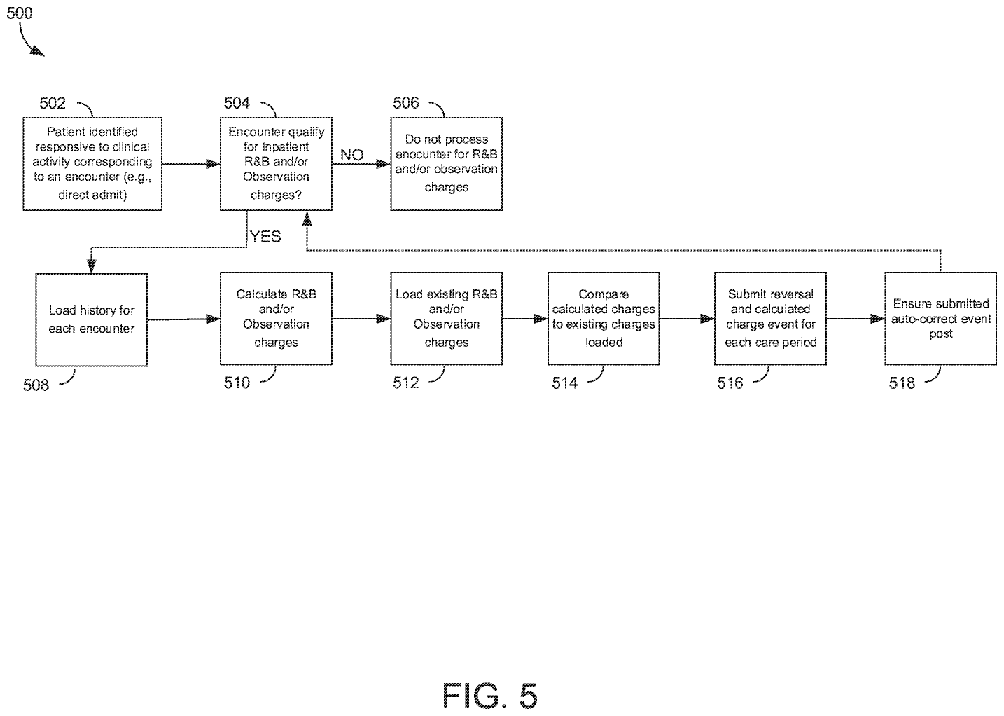

Turning now to FIG. 5, a flow diagram is provided illustrating a method 500 for auto correction of a charge event (e.g., auto correction of a room and bed and/or observation charge) utilizing an auto-correction clinical system, in accordance with various embodiments of the present disclosure. The method 500 may be performed by any computing device (such as the computing device described with respect to FIG. 1) with access to an auto-correction system (such as the one described with respect to FIG. 2) or by one or more components of the auto-correction system (such as the auto-correction clinical system, identity circuitry, and/or the intelligence circuitry described with respect to FIGS. 2 and 3).

Initially, as shown at 502, a patient may be identified responsive to clinical activity corresponding to an encounter (e.g., direct admit). At 504, it is determined whether the encounter qualifies for inpatient room and bed and/or observation charges. If the encounter does not qualify for inpatient room and bed and/or observation charges, at 506, the encounter may not be processed for one or more charge events (e.g., one or more inpatient room and bed and/or observation charges). If the encounter qualifies for inpatient room and bed and/or observation charges, the history (e.g., the accommodation history, encounter history, patient events, or any other suitable history) corresponding to the patient associated with the encounter(s) is received or otherwise loaded via the auto-correction clinical system, at 508. The history may be received for each encounter that corresponds to the patient.

The charge event(s) (e.g., the room and bed and/or observation charges) that should be applied for each encounter are calculated by the auto-correction clinical system at 510. The auto-correction clinical system may subtract one or more charge exclusions from one or more charge events, in some embodiments. If the auto-correction clinical system determines a charge event (e.g., a missing charge) is needed for a date of service that does not correspond to clinical activity, the intelligence circuitry may determine the charge event (e.g., an inpatient charge this is missing) from the accommodation history corresponding to the patient status order as described herein with reference to FIGS. 3 and 4.

The existing room and bed and/or observation charge(s)) is received or otherwise loaded at 512, by the auto-correction clinical system. At 514, a calculated charge event may be compared to the existing charge event loaded. In some embodiments, the comparison of charge events may not include one or more care periods (e.g., service dates) associated with indicators of a charge event that includes a manual and/or transformed charge. The comparison of charge events may include one or more care periods associated with indicators of a charge event that includes a manual and/or transformed charge if the encounter status from the patient status order does not align with existing charge event. For example, the auto-correction clinical system may compare the charge event (e.g., a manually entered inpatient room and bed and/or observation charge) determined to the secondary charge event (e.g., one or more existing observation charges). The inpatient room and bed and/or observation charge may be associated with a manual indication responsive to the manual entry of the room and bed and/or observation charge on day 1. If, for example, the encounter status indicates the patient was under observation on day 1, the room and bed charge may be credited and the observation charge may be posted even though the room and bed charge is associated with the manual indication.

At 516, a reversal and/or calculated charge event may be submitted for each care period (e.g., each date of service) by the auto-correction clinical system. A reversal may be submitted for a secondary charge event (e.g., an existing charge) that may not align with the charge event(s) calculated. The auto-correction clinical system may generate an auto-correct event (e.g., a corrected or otherwise new charge) for dates of service that may not correspond to a charge (e.g., dates of service for which a charge does not exist). The submission of the auto-correct event may be confirmed or otherwise ensured by the auto-correction clinical system, at 518.

As can be understood, the present invention provides systems and methods for automatically correcting a charge event (e.g., a room and bed charge or an observation charge) aligned to clinical history. The present invention has been described in relation to particular embodiments, which are intended in all respects to be illustrative rather than restrictive. Alternative embodiments will become apparent to those of ordinary skill in the art to which the present invention pertains without departing from its scope.

From the foregoing, it will be seen that this invention is one well adapted to attain all the ends and objects set forth above, together with other advantages which are obvious and inherent to the system and method. It will be understood that certain features and subcombinations are of utility and may be employed without reference to other features and subcombinations. This is contemplated and within the scope of the claims.

What is claimed is:

1. A method comprising:
receiving, via an auto-correction clinical system comprising intelligence circuitry, accommodation history corresponding to a patient;
receiving, via the auto-correction clinical system, encounter history corresponding to the patient;
determining, via the intelligence circuitry, whether the accommodation history comprises a gap;
determining, via the auto-correction clinical system, a charge event corresponding to a patient status indicator and a care period corresponding to one or more days responsive to the determination of the gap in the accommodation history and the encounter history; and
generating, via the auto-correction clinical system, an auto-correct event based on the charge event and the care period.

2. The method of claim 1, wherein the charge event comprises at least one of a room and bed charge or an observation charge.

3. The method of claim 1, wherein generating the auto-correct event based on the charge event and the care period comprises:
comparing the charge event to a secondary charge event; and
determining the auto-correct event based on the compared charge event and the secondary charge event according to the care period.

4. The method of claim 1, further comprising reversing the charge event corresponding to the patient status indicator.

5. The method of claim 1, wherein the patient status indicator comprises a patient level of care.

6. The method of claim 1, wherein the intelligence circuitry is structured to fill-in at least one of the charge event, the care period, or a patient level of care.

7. The method of claim 1, wherein the auto-correction clinical system is structured to monitor clinical activity, and wherein the clinical activity corresponds to an encounter associated with the patient.

8. The method of claim 7, wherein the encounter is associated with the encounter history corresponding to the patient.

9. Computer-readable storage media having computer-executable instructions embodied thereon that, when executed by one or more processors, cause the processors to:
receive, via an auto-correction clinical system comprising intelligence circuitry, accommodation history corresponding to a patient;
receive, via the auto-correction clinical system, encounter history corresponding to the patient;
determine, via the intelligence circuitry, whether the accommodation history comprises a gap;
determine, via the auto-correction clinical system, a charge event corresponding to a patient status indicator and a care period corresponding to one or more days based on the determination of the gap in the accommodation history; and
generate, via the auto-correction clinical system, an auto-correct event based on the charge event and the care period.

10. The computer-readable storage media of claim 9, wherein the charge event comprises at least one of a room and bed charge or an observation charge.

11. The computer-readable storage media of claim 9, wherein the patient status indicator is associated with clinical activity corresponding to a clinical unit.

12. The computer-readable storage media of claim 11, wherein the clinical activity corresponds to an encounter associated with the patient, and wherein the encounter is associated with the encounter history corresponding to the patient.

13. The computer-readable storage media of claim 11, wherein the accommodation history is received in response to the clinical activity.

14. The computer-readable storage media of claim 9, wherein the patient status indicator comprises a patient level of care.

15. A system comprising:
one or more processors; and
a non-transitory computer storage media storing computer-useable instructions that, when used by the one or more processors, cause the one or more processors to:
receive, via an auto-correction clinical system comprising intelligence circuitry, accommodation history corresponding to a patient;
receive, via the auto-correction clinical system, encounter history corresponding to the patient;
determine, via the intelligence circuitry, whether the accommodation history comprises a gap;
determine, via the auto-correction clinical system, a charge event corresponding to a patient status indicator and a care period corresponding to one or more days responsive to the determination of the gap in the accommodation history and the encounter history; and
generate, via the auto-correction clinical system, an auto-correct event based on the charge event and the care period.

16. The system of claim 15, wherein the charge event comprises at least one of a room and bed charge or an observation charge.

17. The system of claim 15, wherein the charge event is split across multiple units within the care period.

18. The system of claim 15, wherein the patient status indicator is associated with clinical activity corresponding to a clinical unit.

19. The system of claim 18, wherein the auto-correction clinical system is structured to monitor the clinical activity, and wherein the clinical activity corresponds to an encounter associated with the patient.

20. The system of claim 19, wherein the encounter is associated with the encounter history corresponding to the patient.
